SEQUENCE LISTING (12 sequences)

SEQ ID NO: 1 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 2 - DNA, 1755 bp, Homo sapiens
acggagatctcgccggctttacgttcacctcggtgtctgcagcaccctccgcttcctctcctaggcgacgagacccagtggctagaagttcaccatgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagcttttgcagtcgtgtaatgggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgag

SEQ ID NO: 3 - protein, 2413 aa, Homo sapiens
MGISTVILEMCLLWGQVLSTGGWIPRTTDYASLIPSEVPLDPTVAEGSPFPSESTLESTVAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYRGSWGTVCDDSWDTNDANVVCRQLGCGWAMLAPGNARFGQGSGPIVLDDVRCSGNESYLWSCPHNGWLSHNCGHSEDAGVICSGPESSLALRLVNGGDRCQGRVEVLYRGSWGTVCDDSWDTNDANVVCRQLGCGWATSAPGNARFGQGSGPIVLDDVRCSGHESYLWSCPNNGWLSHNCGHHEDAGVICSAAQSRSTPRPDTLSTITLPPSTVGSESSLTLRLVNGSDRCQGRVEVLYRGSWGTVCDDSWDTNDANVVCRQLGCGWATSAPGNARFGQGSGPIVLDDVRCSGHESYLWSCPHNGWLSHNCGHHEDAGVICSVSQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQLGCGWAMSAPGNARFGQGSGPIVLDDVRCSGHESYLWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 4 - DNA, 5802 bp, Homo sapiens
tttatagcagcagcagaaatataccaccctagaggacacacctccttttagctaggtacctataaatgtccaggattttctattcaattgagaagaacccagcaaaatggggatctccacagtcatccttgaaatgtgtcttttatggggacaagttctatctacaggtgggtggatcccaaggactacagactacgcttcactgattccctcggaggtgcccttggatcaaactgtagcagaaggttctccatttccctcggagtcgaccctggagtcaactgcagcagaaggttctccgatttccttggagtcaaccctggagtcaactgtagcagaaggttctctgattccctcagagtcaaccctggagtcaactgtagcagaaggatctgattctggtttggccctgaggctggtgaatggagatggcaggtgtcagggccgagtggagatcctataccgaggctcctggggcaccgtgtgtgatgacagctgggacaccaatgatgccaacgtggtctgtaggcagctgggttgtggctgggccatgtcagctccaggaaatgcctggtttggccagggctcaggacccattgccctggatgatgtgcgctgctcaggacacgaatcctacctgtggagctgcccccacaatggctggctctcccataactgtggccatggtgaagatgctggtgttatctgctcagctgcccagcctcagtcaacactcaggccagaaagttggcctgtcaggatatcaccacctgtacccacagaaggatctgaatccagtttggccctgaggctggtgaatggaggcgacaggtgtcgaggccgagtggaggtcctataccgaggctcctggggcaccgtgtgtgatgactactgggacaccaatgatgccaatgtggtctgcaggcagctgggctgtggctgggccatgtcagccccaggaaatgcccagtttggccagggctcaggacccattgtcctggatgatgtgcgctgctcaggacacgagtcctacctgtggagctgcccccacaatggctggctcacccacaactgtggccatagtgaagacgctggtgtcatctgctcagctccccagtcccggccgacacccagcccagatacttggccgacctcacatgcatcaacagcaggacctgaatccagtttggccctgaggctggtgaatggaggtgacaggtgtcagggccgagtggaggtcctataccgaggctcctggggcaccgtgtgtgatgatagctgggacaccagtgacgccaatgtggtctgccggcagctgggctgtggctgggccacgtcagccccaggaaatgcccggtttggccagggttcaggacccattgtcctggatgacgtgcgctgctcaggctatgagtcctacctgtggagctgcccccacaatggctggctctcccataactgtcagcacagtgaagacgctggtgtcatctgctcagctgcccactcctggtcgacgcccagtccagacacattgccgaccatcaccttgcctgcatcgacagtaggatctgaatccagtttggccctgaggctggtgaatggaggtgacaggtgtcagggccgagtggaggtcctataccaaggctcctggggcaccgtgtgcgatgacagctgggacaccaatgatgccaatgtcgtctgcaggcaaccgggctgtggctgggccatgtcagccccaggaaatgcccggtttggtcagggctcaggacccattgtcctggatgatgtgcgctgctcaggacacgagtcttacccgtggagctgcccccacaatggctggctctcccacaactgtggccatagtgaagacgctggtgtcatctgctcagcttcccagtcccggccaacacctagtccagacacttggccaacctcacatgcatcaacagcaggatctgaatccagtttggccctgaggctggtgaatggaggtgacaggtgtcagggccgagtggaggtcctataccgaggctcctggggcaccgtgtgtgatgactactgggacaccaatgatgccaatgtggtttgcaggcagctgggctgtggctgggccatgtcagccccaggaaatgcccggtttggccagggttcaggacccattgtcctggatgatgtgcgctgctcaggacatgagtcctatctgtggagctgcccccacaatggctggctctcccacaactgtggccatcatgaagacgctggtgtcatctgctcagcttcccagtcccagccgacacccagcccagacacttggccaacctcacatgcatcaacagcaggatctgaatccagtttggccctgaggctggtgaatggaggtgacaggtgtcagggccgagtggaggtcctataccgaggctcctggggcaccgtgtgtgatgactactgggacaccaatgatgccaatgtggtttgcaggcagctgggctgtggctgggccacgtcagccccaggaaatgcccggtttggccagggttcaggacccattgtcctggatgatgtgcgctgctcaggacatgagtcctatctgtggagctgcccccacaatggctggctctcccacaactgtggccatcatgaagacgctggtgtcatctgctcagcttcccagtcccagccgacacccagcccagacacttggccaacctctcgtgcatcaacagcaggatctgaatccactttggccctgagactggtgaatggaggtgacaggtgtcgaggccgagtggaggtcctataccaaggctcctggggcaccgtgtgtgatgactactgggacaccaatgatgccaacgtggtctgcaggcagctgggctgtggctgggccatgtcagccccaggaaatgcccagtttggccagggctcaggacccattgtcctggatgatgtgcgctgctcaggacacgagtcttacctgtggagctgcccccacaatggctggctctcccacaactgtggccatcatgaagatgctggtgtcatctgctcagctgctcagtcccagtcaacgcccaggccagatacttggctgaccaccaacttaccggcattgacagtaggatctgaatccagtttggctctgaggctggtgaatggaggtgacaggtgtcgaggccgagtggaggtcctgtatcgaggctcctggggaaccgtgtgtgatgacagctgggacaccaatgatgccaatgtggtctgcaggcagctgggctgtggctgggccatgtcggccccaggaaatgcccggtttggccagggctcaggacccattgtcctggatgatgtgcgctgctcagggaatgagtcctacctgtggagctgcccccacaaaggctggctcacccacaactgtggccatcacgaagacgctggtgtcatctgctcagccacccaaataaattctactacgacagattggtggcatccaacaactacaaccactgcaagaccctcttcaaattgtggtggcttcttattctatgccagtgggacattctccagcccatcctaccctgcatactaccccaacaatgctaagtgtgtttgggaaatagaagtgaattctggttatcgcataaacctgggcttcagtaatctgaaattggaggcacaccataactgcagttttgattatgttgaaatctttgatggatcattgaatagcagtctcctgctggggaaaatctgtaatgataccaggcaaatatttacatcttcttacaaccgaatgaccattcactttcgaagtgacatcagtttccaaaacactggctttttggcttggtataactccttcccaagcgatgccaccttgaggttggtcaatttaaattcatcctatggtctatgtgccgggcgtgtagaaatttaccatggtggcacctgggggacagtttgtgatgactcctggaccattcaggaagctgaggtggtctgcagacagctagggtgtggacgtgcagtttcagcccttggaaatgcatattttggctctggctctggccccatcaccctggacgatgtagagtgctcagggacggaatccactctctggcagtgccggaaccgaggctggttctcccacaactgtaatcatcgtgaagatgctggtgtcatctgctcaggaaaccatctatcgacacctgctccttttctcaacatcacccgtccaaacacagattattcctgcggaggcttcctatcccaaccatcaggggacttttccagcccattctatcccgggaactatccaaacaatgccaagtgtgtgtgggacattgaggtgcaaaacaactaccgtgtgactgtgatcttcagagatgtccagcttgaaggtggctgcaactatgattatattgaagttttcgatggcccctaccgcagttcccctctcattgctcgagtttgtgatggggccagaggctccttcacttcttcctccaacttcatgtccattcgcttcatcagtgaccacagcatcacaaggagagggttccgggctgagtactactccagtccctccaatgacagcaccaacctgctctgtctgccaaatcacatgcaagccagtgtgagcaggagctatctccaatccttgggcttttctgccagtgaccttgtcatttccacctggaatggatactacgagtgtcggccccagataacgccgaacctggtgatattcacaattccctactcaggctgcggcaccttcaagcaggcagacaatgacaccatcgactattccaacttcctcacagcagctgtctcaggtggcatcatcaagaggaggacagacctccgtattcacgtcagctgcagaatgcttcagaacacctgggtcgacaccatgtacattgctaatgacaccatccacgttgctaataacaccatccaggtcgaggaagtccagtatggcaattttgacgtgaacatttccttttatacttcctcatctttcttgtatcctgtgaccagccgcccttactacgtggacctgaaccaggacttgtacgttcaggctgaaatcctccattctgatgctgtactgaccttgtttgtggacacctgcgtggcatcaccatactccaatgacttcacgtctttgacttatgatctaatccggagtggatgcgtgagggatgacacctacggaccctactcctcgccgtctcttcgcattgcccgcttccggttcagggccttccacttcctgaaccgcttcccctccgtgtacctgcgttgtaaaatggtggtgtgcagagcgtatgacccctcttcccgctgctaccgaggctgtgtgttgaggtcgaagagggatgtgggctcctaccaggaaaaggtggacgtcgtcctgggtcccatccagctgcagacccccccacgccgagaagaggagcctcggtaggtggtcgctctcagaccccactgtccaccggggcgcagacccctgactcggggacttgggatgttcctcttggtgtcatattccaactcagattgagccctacattgtgctgcacctggtcatacggagttgaatcagacctggttcccgcctcccccaaggctcatggtccttggaggacccgttgcagggcgaggtcaagagagttctgacctggatggcccatagacctgacgtcccagaatccatgcttctcatctgcaaaatgaaaatgtcaatacttacttcttagcactgttgagagggttacttacataaaggaattttggtgaaactgc

SEQ ID NO: 5 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 6 - DNA, 1318 bp, Homo sapiens
ggagacgcacgcagctgactttgtcttctccgcacgactgttacagaggtctccagagccttctctctcctgtgcaaaatggcaactcttaaggaaaaactcattgcaccagttgcggaagaagaggcaacagttccaaacaataagatcactgtagtgggtgttggacaagttggtatggcgtgtgctatcagcattctgggaaagtctctggctgatgaacttgctcttgtggatgttttggaagataagcttaaaggagaaatgatggatctgcagcatgggagcttatttcttcagacacctaaaattgtggcagataaagattattctgtgaccgccaattctaagattgtagtggtaactgcaggagtccgtcagcaagaaggggagagtcggctcaatctggtgcagagaaatgttaatgtcttcaaattcattattcctcagatcgtcaagtacagtcctgattgcatcataattgtggtttccaacccagtggacattcttacgtatgttacctggaaactaagtggattacccaaacaccgcgtgattggaagtggatgtaatctggattctgctagatttcgctaccttatggctgaaaaacttggcattcatcccagcagctgccatggatggattttgggggaacatggcgactcaagtgtggctgtgtggagtggtgtgaatgtggcaggtgtttctctccaggaattgaatccagaaatgggaactgacaatgatagtgaaaattggaaggaagtgcataagatggtggttgaaagtgcctatgaagtcatcaagctaaaaggatataccaactgggctattggattaagtgtggctgatcttattgaatccatgttgaaaaatctatccaggattcatcccgtgtcaacaatggtaaaggggatgtatggcattgagaatgaagtcttcctgagccttccatgtatcctcaatgcccggggattaaccagcgttatcaaccagaagctaaaggatgatgaggttgctcagctcaagaaaagtgcagataccctgtgggacatccagaaggacctaaaagacctgtgactagtgagctctaggctgtagaaatttaaaaactacaatgtgattaactcgagcctttagttttcatccatgtacatggatcacagtttgctttgatcttcttcaatatgtgaatttgggctcacagaatcaaagcctatgcttggtttaatgcttgcaatctgagctcttgaacaaataaaattaactattgtagtgtgaaaaaaaaaaaaaaaaaaaaaaaaaaaaaaaaaaa

SEQ ID NO: 7 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 8 - DNA, 1031 bp, Homo sapiens
attcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaa

SEQ ID NO: 9 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 10 - DNA, 2379 bp, Homo sapiens
cctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccggcatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggttctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtggattaggggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattcaaaaaaaaaaaaaaaaaa

SEQ ID NO: 11 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 12 - DNA, 789 bp, Homo sapiens
gcccagcgccccgcacttttctgagcagacgtccagagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcccggatgcccacccctgctgccgccactggctgtgcctcccccgccacctgtgtgttcttttgatacatttatcttctgtttttctcaaataaagttcaaagcaaccacc

(19) United States
(12) Patent Application Publication
(10) Pub. No.: US 2016/0223537 A1
(43) Pub. Date: Aug. 4, 2016

(54) IDENTIFICATION OF NOVEL BIOMARKERS OF FLARES OF SYSTEMIC LUPUS ERYTHEMATOSUS

(71) Applicant: SINGAPORE HEALTH SERVICES PTE LTD, Singapore (SG)

(72) Inventors: KOK YONG FONG, SINGAPORE (SG); JULIAN THUMBOO, SINGAPORE (SG); SZU-TIEN THIO, SINGAPORE (SG); HAK TIEN LOUISE-MARIE GRIGNION TONG, SINGAPORE (SG); LEI ZHOU, SINGAPORE (SG)

(73) Assignee: SINGAPORE HEALTH SERVICES PTE LTD, SINGAPORE (SG)

(21) Appl. No.: 15/021,225

(22) PCT Filed: Sep. 11, 2014
(86) PCT No.: PCT/SG2014/000428

§ 371 Date: Mar. 10, 2016

(30) Foreign Application Priority Data
Sep. 11, 2013 (SG) 201306892-9

(51) Int. Cl. G01N 33/564; C12Q 1/68

(52) U.S. Cl. CPC G01N 33/564; C12Q 1/6883; G01N 2800/104; G01N 2800/52; C12Q 2600/118; C12Q 2600/158

(57) ABSTRACT

The present invention relates to methods for diagnosing or monitoring the flare status of subjects with Systemic Lupus Erythematosus (SLE). More particularly, the present invention relates to testing fluid samples from subjects for differentially expressed biomarkers, and kits for performing the tests.

### FIELD OF INVENTION

The present invention relates generally to methods for diagnosing or monitoring the flare status of subjects with Systemic Lupus Erythematosus (SLE). More particularly, the present invention relates to screening samples from subjects for particular protein biomarkers which have efficacy in identifying or predicting a flare.

### BACKGROUND

The prevalence of Systemic Lupus Erythematosus (SLE) ranges from 40/100,000 cases for Northern Europeans to 200/100,000 cases in Blacks. There are more than 250,000 patients with SLE in the United States. The 15-year survival rate is 80-85% and the chance of dying by 35 years of age from lupus or infection is 1 in 6 patients.

Currently available biomarkers for SLE flares (dsDNA, C3, C4) are useful in detecting flares in 60-70% of SLE patients, but do not detect flares in a substantial number of patients, including those with major organ involvement e.g. lupus nephritis. They are therefore of limited value for routine clinical use. These biomarkers also require blood draws which are associated with discomfort for patients. Frequent, regular monitoring of biomarkers is important in predicting incipient flares in SLE. There is therefore an unmet need and tear biomarkers, if validated, may fulfill this role as the collection is non-invasive and more convenient in comparison with blood draws. As some tear proteins are involved in immune system functioning, the study of potential biomarkers in tears is a novel area of research in autoimmune disease.

Flares occur in approximately 80% of patients during the course of their disease [Petri M, et al., Am J Med 1991; 91: 345-54], and generally require the introduction or increase in dose of a variety of potentially toxic therapies. The morbidity and mortality associated with flares can be substantial, and is related to organ damage resulting from active SLE per se and the adverse effects of corticosteroids and immunosuppressive drugs [Abu-Sharaka M, et al., J Rheumatol 1995; 22: 1259-64]. In SLE patients with nephritis, for example, doubling of serum creatinine was 7 times more likely in patients with a renal flare, and 27 times more likely in severe renal flares manifesting as nephritic syndrome [Petri M, et al., Arthritis Rheum 1991; August 34(8): 937-44]. Flares also result in significant mortality, accounting for 26% of deaths within 5 years of diagnosis and 10% of deaths more than 5 years after diagnosis in one series [Abu-Sharaka M, et al., J Rheumatol 1995; 22: 1259-64]. Flares of disease activity often necessitate the use of corticosteroids and/or immunosuppressives. Corticosteroid use may lead to osteoporosis, infections (at times fatal) and avascular necrosis of bone [Ehrenstein M R, et al., Br J Rheumatol 1995; 34(3): 257-60]. Immunosuppressive therapy is also associated with significant morbidity and mortality. For example, 27% of 127 SLE patients treated with cyclophosphamide in Singapore developed major infections, with 4 patients perishing from these infections [Mirzayan M J, et al., Rheumatology (Oxford) 2000; 39(12): 1316-9].

Flares of SLE disease activity (a major source of morbidity/mortality) are inadequately predicted by existing biomarkers. As some tear and saliva proteins are involved in immune system functioning, the study of potential biomarkers in tears and saliva is a novel area of research in autoimmune disease. We used iTRAQ with nano LC-MS/MS [Zhou L, et al., J Proteome Res 2009; 8: 4889-905] to monitor changes in the levels of various potential protein biomarkers in serial tear and saliva specimens from SLE patients.

### SUMMARY OF THE INVENTION

The expression signatures identified in this study had sufficient diagnostic efficacy for development into tear- and saliva-based biomarkers for diagnosing or monitoring the flare status in subjects with SLE.

Accordingly, in a first aspect, the present invention provides a method of analysing a fluid test sample from a subject, comprising a step of determining the level of at least one biomarker in the test sample and comparing said level to that in a reference sample, wherein the at least one biomarker is selected from the group comprising Enolase 1 (ENO1), Deleted in Brain Tumour 1 (DMBT1), Lactate dehydrogenase A (LDHA) or Lactate dehydrogenase B (LDHB) and Heat Shock 70 kDa Protein 1B (HSPA1B) and Heat Shock 27 kDa Protein 1 (HSPB1). In a preferred embodiment of the invention, the at least one biomarker comprises DMBT1.

Another aspect of the invention provides a method of diagnosing or monitoring the flare status of SLE in a subject, comprising screening a fluid test sample from the subject for the presence of at least one biomarker differentially expressed in a flare state compared to a reference sample, wherein the at least one biomarker is selected from the group consisting of ENO1, DMBT1, LDHA, LDHB, HSPA1B and HSPB1; and

diagnosing or monitoring the status of SLE in the subject, based on expression of the at least one biomarker.

In a preferred embodiment of the invention, the test sample is selected from the group comprising tear, saliva, blood and urine.

In a preferred embodiment of the invention, the reference sample represents the biomarker expression profile of a normal subject or an SLE subject in a quiescent or flare state.

In another preferred embodiment of the invention, the reference sample represents the biomarker expression profile of an SLE subject in a quiescent state.

In another preferred embodiment of the invention, the status of SLE in a subject is incipient flare or disease activity.

In another preferred embodiment of the invention, the expression of the at least one biomarker is detected using tandem Mass Spectrometry, using antibodies directed to said biomarker in immunoassay.

In another aspect of the invention, the flare status of SLE in a subject comprises response to a treatment administered to the subject.

Another aspect of the invention provides a SLE diagnostic or monitoring kit comprising at least one probe which specifically binds to at least one biomarker selected from the group consisting of ENO1, DMBT1, LDHA, LDHB, HSPA1B and HSPB1.

### BRIEF DESCRIPTION OF THE FIGURES

FIG. 1: Flow chart of study subjects and study enrollment.

FIG. 2: Sampling of tear specimens for subsets of patients.

FIG. 3: Experimental design for iTRAQ proteomics.

FIG. 4: Ratio Proteomic Profile: Flare vs non-Flare average from 8 patients.

FIG. 5: Log Ratio Proteomic Profile: Flare vs non-Flare average from 0.8 patients.

FIG. 6: Numbers 1˜16 in (A) and (B) indicate 16 flare states (from 8 patients, each patient has 2 flare states). Numbers 1˜8 in (C) and (D) indicate 8 non-flare states (from 8 patients, each patient has 1 non-flare state). Dark: protein level indicates the flare state. Light: protein level fails to indicate the flare state. (A): Combination of DMBT1 and LDHB, ratio >1.5 or <0.67, 15/16 (93.8%) correct; (B): Combination of DMBT1 and ENO1, ratio >1.5 or <0.67, 16/16 (100%) correct; (C): Combination of DMBT1 and LDHB, ratio <1.5 or >0.67, 8/8 (100%) correct whereas if use single biomarker of DMBT1 resulted in 6/8 (75%) correct and LDHB resulted in 4/8 (50%) correct; (D): Combination of DMBT1 and ENO1, ratio <1.5 or >0.67, 8/8 (100%) correct whereas if use single biomarker of either DMBT1 or ENO1 resulted in 6/8 (75%) correct.

FIG. 7: Tear DMBT1 levels in two individual patients with SLE in pre-flare, flare and post-flare states. (1 and 2: pre-Flare, 3 and 4: Flare, 5, 6 and 7: post-Flare).

### DETAILED DESCRIPTION

Although various body fluids may be tested for the presence of flare biomarkers, the collection of tears, urine and saliva is relatively non-invasive as compared to withdrawal of blood. Such tear, urine and saliva sampling would allow for more regular collection of specimens, even by the patient themselves if a suitable sampling and testing kit were available. If a patient could test their own tears, urine or saliva for biomarkers indicative of a flare state, they would have a stronger incentive to follow up with further analysis by seeing a doctor for assessment of SLE flare status. Frequent regular monitoring of biomarkers is likely to be important in predicting incipient flares, as the levels of such biomarkers may rise or fall relatively proximately to a flare.

### DEFINITIONS

Certain terms employed in the specification, examples and appended claims are collected here for convenience.

An SLE flare is defined herein as either SLEDAI score 4 or physician assessed flares. The rationale for including physician assessed flares is that the SLEDAI does not cover some rarer manifestations of flares, e.g. gut vasculitis, new onset of peripheral neuropathy.

As used herein, ‘incipient flare’ means the sub-clinical, beginning, early or emerging stages of a flare.

As used herein, ‘quiescent state’ means the subject is not experiencing a clinical flare, in the presence or absence of serological indications. We do note that 10% of SLE patients are “clinically quiescent, biochemically active” (i.e. no clinical flare though existing biomarkers suggest the presence of a flare) and another 10% of SLE patients are “clinically active, biochemically quiescent” (i.e. clinical flare though existing biomarkers do not suggest the presence of a flare) and that the proposed patent can help to clarify the clinical state of these patients.

As used herein, ‘biomarker’ refers to a protein molecule or nucleic acid which is differentially expressed in the tears and/or saliva during the quiescent state compared to flare state in SLE patients. According to the invention one or more biomarkers may be selected from Enolase 1 (ENO1), Deleted in Brain Tumour 1 (DMBT1), Lactate dehydrogenase A (LDHA), Lactate dehydrogenase B (LDHB), Heat Shock 70 kDa Protein 1B (HSPA1B) and Heat Shock 27 kDa Protein 1 (HSPB1).

A representative amino acid sequence of human ENO1 is shown in SEQ ID NO: 1, and nucleic acid sequence shown in SEQ ID NO: 2. A representative amino acid sequence of human DMBT1 is shown in SEQ ID NO: 3, and nucleic acid sequence shown in SEQ ID NO: 4. A representative amino acid sequence of human LDHA is shown in SEQ ID NO: 5, and nucleic acid sequence shown in SEQ ID NO: 6. A representative amino acid sequence of human LDHB is shown in SEQ ID NO: 7, and nucleic acid sequence shown in SEQ ID NO: 8. A representative amino acid sequence of human HSPA1B is shown in SEQ ID NO: 9, and nucleic acid sequence shown in SEQ ID NO: 10. A representative amino acid sequence of human HSPB1 is shown in SEQ ID NO: 11 and nucleic acid sequence shown in SEQ ID NO: 12.

An antibody is any immunoglobulin, including antibodies and fragments thereof that bind to a specific epitope. An antibody for use in the invention may be prepared against an isolated or recombinant form of a biomarker protein of the invention. Such antibodies include, but are not limited to polyclonal, monoclonal, chimeric, humanised, single chain, Fab, Fab′, F(ab)′ fragments and/or F(v) portions of the whole antibody which are capable of binding to a biomarker to enable its identification and quantitation. The polypeptide or oligopeptide used to immunize an animal (e.g., a mouse, a rat, or a rabbit) can be derived from the translation of RNA, an expression construct such as a plasmid, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal. Suitable biomarker detection antibodies may also be commercially available.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay. As used herein, the term “oligonucleotide” is substantially equivalent to the terms “amplimers,” “primers,” “oligomers,” and “probes,” as these terms are commonly defined in the art.

The term “comprising” as used in the context of the invention refers to where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the biomarker of the present invention “substantially” comprises the indicated protein as “essential” element. Additional markers may be included in the methods and kits of the invention.

One aspect of the present invention provides a method of analysing a fluid test sample from a subject, comprising a step of determining the level of at least one biomarker in the test sample and comparing said level to that in a reference sample, wherein the at least one biomarker is selected from the group comprising Enolase 1 (ENO1), Deleted in Brain Tumour 1 (DMBT1), Lactate dehydrogenase A (LDHA) or Lactate dehydrogenase B (LDHB) and Heat Shock 70 kDa Protein 1B (HSPA1B) and Heat Shock 27 kDa Protein 1 (HSPB1).

In a preferred embodiment of the invention, the at least one biomarker comprises DMBT1. Preferably the at least one biomarker comprises DMBT1 and LDHB or DMBT1 and ENO1. The studies described herein show that these pairs of biomarkers increased the accuracy of diagnosis to 100%.

Preferably the subject is a human.

Preferably, the test sample and the reference sample isolated from the subject are fluid samples, such as from tears, saliva, blood or urine. More preferably the sample is a tear or saliva sample. Most preferably the sample is a tear sample.

Another aspect of the invention provides a method of diagnosing or monitoring the flare status of SLE in a subject, comprising screening a fluid test sample from the subject for the presence of at least one biomarker differentially expressed in a flare state compared to a reference sample, wherein the at least one biomarker is selected from the group consisting of ENO1, DMBT1, LDHA, LDHB, HSPA1B and HSPB1; and

diagnosing or monitoring the status of SLE in the subject based on expression of the at least one biomarker.

In a preferred embodiment of the invention, the reference sample represents the biomarker expression profile of a normal subject or an SLE subject in a quiescent or flare state.

In another preferred embodiment of the invention, the reference sample represents the biomarker expression profile of an SLE subject in a quiescent state.

In another preferred embodiment of the invention, the status of SLE in a subject is incipient flare or disease activity.

In another preferred embodiment of the invention, the expression of the at least one biomarker is detected using tandem Mass Spectrometry, using antibodies directed to said biomarker or using a nucleic acid technology.

A suitable method and equipment to perform Mass Spectrometry is described in the examples herein.

Suitable antibodies may be generated using conventional methods, including animal immunization with an isolated or recombinant biomarker protein or antigenic fragment thereof. A suitable isolated or recombinant biomarker protein may have the sequence set forth in SEQ ID NO: 1, SEQ ID NO: 3, SEQ ID NO: 5, SEQ ID NO: 7, SEQ ID NO: 9 or SEQ ID NO: 11. Isoforms or splice variants of these proteins and biomarker proteins from other species which are homologous to the aforementioned biomarkers may also be suitable as antigens to generate antibodies for use according to the invention.

The biomarker proteins of the invention may be produced for immunization purposes synthetically or via expression constructs which encode them. An example of a suitable expression vector is the bacterial plasmid pGEX-4T-1, which encodes a fusion protein with a thrombin cleavage site and is available from GE Healthcare Bio-Sciences Corp, NJ, USA.

Alternatively, commercial antibodies to the said biomarkers may be used. For example, mouse anti human DMBT1 monoclonal antibody [HYB 213-01-02]; mouse anti-alpha-enolase (anti-ENO1) antibody, monoclonal [MA5-17627]; mouse anti-L-lactate dehydrogenase B chain antibody, monoclonal [MA5-17242]; mouse anti-L-lactate dehydrogenase A chain antibody, monoclonal [MA5-17247]; and mouse anti-Heat shock protein beta-1 antibody, monoclonal [G3.1] are available from Thermo Fisher Scientific, MA, USA.

In another preferred embodiment of the invention, the nucleic acid technology further comprises hybridization or amplification in a quantitative real-time polymerase chain reaction.

In another preferred embodiment of the invention, detecting expression of the at least one biomarker further comprises isolating RNA from a subject sample.

In another preferred embodiment of the invention, detecting expression comprises using at least one primer or probe set to detect the expression of each of the at least one biomarker. The at least one primer or probe set may be based on one or more of the nucleic acid sequences set forth in SEQ ID NO: 2, SEQ ID NO: 4, SEQ ID NO: 6, SEQ ID NO: 8, SEQ ID NO: 10, or SEQ ID NO: 12 or variants or homologues thereof.

In another aspect of the invention, the primers or probe sets comprise a diagnostic kit.

In another aspect of the invention, the status of SLE in a subject comprises response to a treatment administered to the subject. The treatment may be administration of one or more from the group comprising NSAIDS, corticosteroids, antimalarials (such as hydroxychloroquine) and immunosuppressive drugs.

Another aspect of the invention provides a SLE flare diagnostic or monitoring kit comprising at least one probe which specifically binds to at least one biomarker selected from the group consisting of ENO1, DMBT1, LDHA, LDHB, HSPA1B and HSPB1.

The kit may comprise one or more antibodies specific to each of the at least one biomarker protein. The kit may also contain suitable solid supports and reagents for western blot, dot blot, ELISA, lateral flow assay (LFA) or any other immunoassay platform using various labeling techniques such as enzyme amplification, radioactivity or fluorescence. An advantage of using ELISA assay is that it is an established and sensitive technique to quantify analytes. ELISA methods are, for example, described in Thermo Scientific Pierce Assay Development Technical Handbook (thermoscientific.com/pierce), incorporated herein by reference. LFA is cheap and easy to use and provides results in 15-20 minutes. LFA is a well-known immunoassay platform, an example of which is described in Lee L G, et al., Biosensors 2013, 3, 360-373, incorporated herein by reference.

Preferably the kit has components suitable for the detection of DMBT1 and ENO1, or DMBT1 and LDHB.

According to any aspect of the present invention, existing SLE biomarkers (e.g. dsDNA, complement levels) may additionally be tested concurrently or sequentially from blood samples and their results combined with the results of the present invention to increase the accuracy of detecting flares of SLE.

A person skilled in the art will appreciate that the present invention may be practiced without undue experimentation according to the methods given herein. The methods, techniques and chemicals are as described in the references given or from protocols in standard biotechnology and molecular biology text books, such as described in Sambrook and Russell, Molecular Cloning: A Laboratory Manual, Cold Springs Harbor Laboratory, New York (2001).

### EXAMPLES

We hypothesised that there are biomarkers in fluid samples of SLE patients which are indicative of flare status. We tested this hypothesis on tear samples by using mass spectroscopy to identify changes in the levels of various potential biomarkers in serial tear specimens from SLE patients.

### Study Design

In this IRB approved study, we prospectively studied 100 subjects with SLE selected from an already assembled cohort of well characterised SLE patients (n=350) in the Department of Rheumatology and Immunology, Singapore General Hospital. Subjects were assessed monthly for 12 months. Additionally, if a patient had a flare of SLE during this period, he/she was reviewed 2 weeks after the flare.

### Experimental Approaches

All enrolled subjects had monthly assessments of:
SLE disease activity measured using SLEDAI 2000, MEX-SLEDAI and Classic BILAG
Presence/severity of dry eyes with monthly Schirmer's test
ACR Damage Index is used to record any damage at the 1^{st} and last study visit
Medication use including eye drops
Information on infection or change in treatment including use of Traditional Chinese Medicine since the previous visit

### Inclusion and Exclusion Criteria:

Inclusion criteria were age 16 years and above, a diagnosis of SLE (fulfilling 4 of 11 American College of Rheumatology 1997 criteria); ability to give written informed consent. For patients 16 to 20 years, informed consent was obtained from the legally acceptable representative (including spouse, parent and guardian).

Exclusion criteria: Inability or refusal to give written informed consent.

### Cohort Characteristics

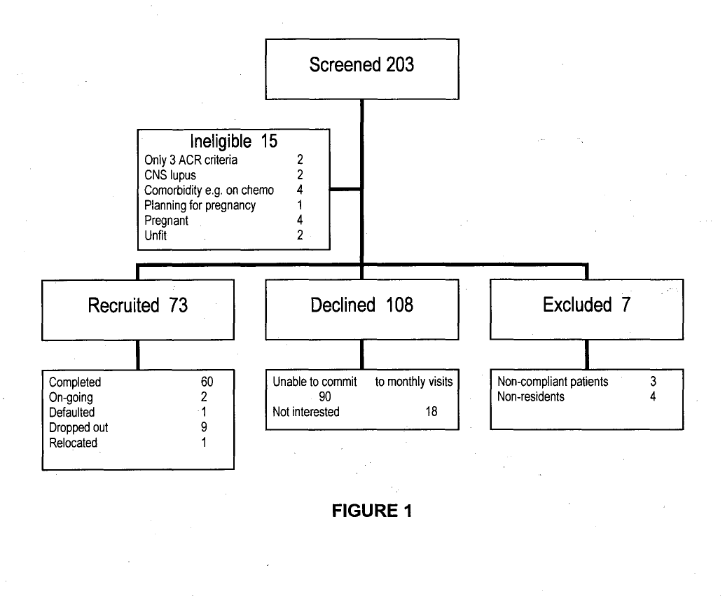

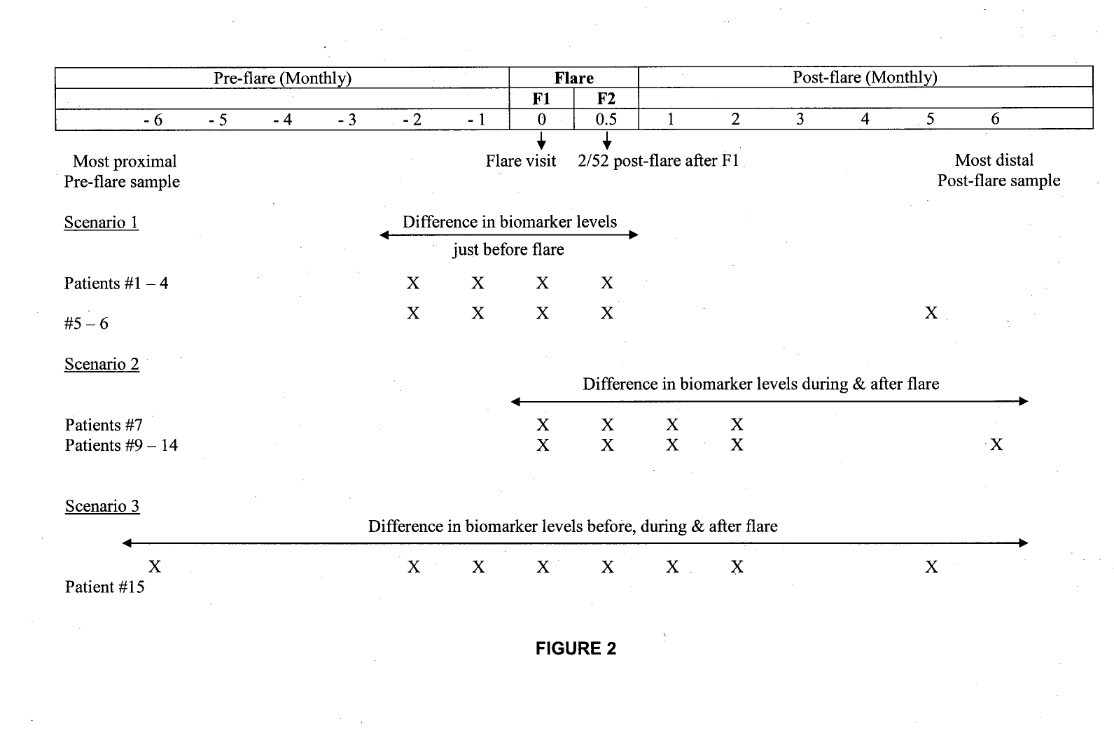

203 subjects were screened, 108 declined, 7 excluded and 15 were ineligible, leaving 73 subjects recruited (FIG. 1).

Of 73 patients with SLE, 14 patients had flares in the study period. The demographic information of these patients is provided in Table 1.

TABLE 1
Characteristics of patients

Patients, n = 14

| | n | % |
|---|---|---|
| Female gender | 10 | 71.4 |
| Ethnic group, | | |
| Chinese | 8 | 57.1 |
| Malay | 3 | 21.4 |
| Indian | 2 | 14.3 |
| Others | 1 | 7.1 |
| Median age (range) at flare, yrs | 37.6 | 23.8-53.9 |
| Median duration (range) of SLE at flare, yrs | 6.1 | 0.0-17.8 |
| SLEDAI ≧ 4 | 13 | 92.9 |
| SLEDAI Median (range) score at flare | 11.5 | 2-31 |

Among the study patients, tear samples were collected at various times before, during and after flares, forming 3 groups of patients as illustrated in FIG. 1. Thus a total of 107 samples from subjects and 20 samples from controls were analysed. The organ involvement of subjects at flare was determined and is shown in Table 2.

TABLE 2
Organ involvement of patients at flare

| | | | | | BILAG | | | | | | | | |
|---|---|---|---|---|---|---|---|---|---|---|---|---|---|
| | | | Age at | SLEDAI | global | | | | | | | | |
| Patient | Gender | Ethnicity | flare (yr) | score | score | GEN | MUC | NEU | MSK | CAR | VAS | REN | HAE |
| 1 | M | M | 54 | 10 | 13 | E | B | E | D | C | E | A | D |
| 2 | F | I | 44 | 11 | 8 | E | B | E | C | E | E | B | C |
| 3 | F | C | 38 | 5 | 4 | E | D | E | C | E | E | E | B |
| 4 | M | I | 32 | 12 | 10 | D | E | E | D | D | E | A | C |
| 5 | F | C | 58 | 9 | 11 | C | B | E | B | C | C | C | C |
| 6 | F | C | 52 | 6 | 10 | B | B | E | B | E | E | E | C |
| 7 | F | M | 36 | 31 | 25 | B | B | B | B | C | D | A | B |
| 8 | F | C | 32 | 8 | 8 | C | C | E | C | C | E | B | C |
| 9 | M | C | 51 | 20 | 19 | B | C | E | C | C | B | A | C |
| 10 | F | C | 24 | 22 | 14 | E | C | E | E | E | B | A | C |
| 11 | F | C | 50 | 2 | 11 | C | E | A | D | D | D | D | C |
| 12 | F | M | 36 | 16 | 25 | A | B | E | C | D | D | A | B |
| 13 | F | C | 37 | 12 | 10 | B | B | E | B | E | E | E | C |
| 14 | M | O | 37 | 14 | 15 | B | B | E | B | C | C | B | C |

### Methods

Briefly, specimens were collected serially in SLE patients (1) at and after a flare, or (2) when quiescent but likely to subsequently flare. iTRAQ sample preparation was followed by two dimensional nano-LC-nano-ESI-mass spectroscopic analysis to identify possible biomarkers in these specimens.

### Subjects

We studied 2 groups of patients with flares:

(1) Subjects with specimens before, during and after flares—up to 8 time points.
(2) Subjects with specimens during and after flares—up to 5 time points.
For each group we also included a specimen several months removed from the time of the flare to act as a control, i.e. when their disease is not active.

### Instruments/Databases

A brief description of MEXSLEDAI can be found in Guzmán J, et al., J Rheumatol 1992 October; 19(10): 1551-8, incorporated herein by reference. This is a modification of the SLEDAI so that laboratory tests are not included, and was used because the intent of the study was to compare these novel biomarkers with the usual biomarkers of disease activity which are included in the SLEDAI. Different weights are given to various clinical manifestations, some clinical manifestations were added (fatigue, lymphopenia) as per Uribe A G, et al., J Rheumatol 2004 October; 31(10): 1934-40, incorporated herein by reference.

### Sample Preparation

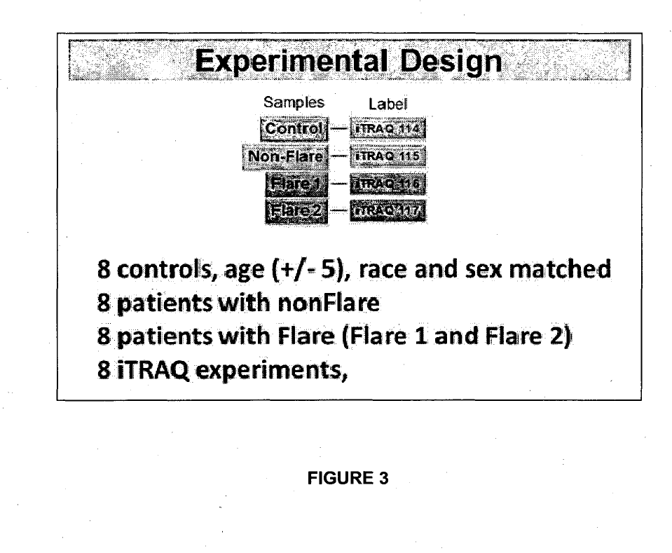

Samples were prepared as described in Lei Zhou et al., Journal of Proteomics 2012; 75: 3877-85. Schirmer strips were cut into small pieces and soaked in 150 μL of 50 mM ammonium bicarbonate solution containing protease inhibitor (Halt protease inhibitor cocktail, Thermoscientific, IL, USA) for 3 hours to elute tear proteins. The protein concentration was measured by colorimetric protein assay (Bio-Rad) using bovine serum album (BSA) as standard. Quantitative proteomics using isobaric tags for relative and absolute quantitation (iTRAQ) technology coupled with 1D-nanoLC-MS/MS was used to identify potential tear protein biomarkers. The experimental design using iTRAQ relative quantitative proteomics technology is illustrated in FIG. 3. Briefly, 50 μg of tear proteins were reconstituted in 50 mM ammonium bicarbonate and reduced by Tris-(2-carboxyethyl) phosphine (TCEP). After 1.5 h of incubation at 60° C., the cysteine residue was blocked by the addition of methyl methanethiosulfonate (MMTS) at room temperature for 20 min. The protein sample was then digested with trypsin (trypsin:protein ratio=1:50) for 16 h at 37° C. The samples were subsequently labeled with iTRAQ reagent (AB SCIEX, Framingham, MA, USA). The samples were then combined and analyzed by one dimensional nanoLC-MS/MS.

One Dimensional nanoLC-MS/MS Proteomic Analysis

The LC-MS/MS was performed using Dionex UltiMate 3000 (Dionex/Thermo Fisher Scientific, Waltham, Mass., USA) coupled with the AB Sciex Triple TOF 5600. For the one dimension separation of peptides, sample was first loaded onto the trap column (Acclaim PepMap 75 mm×2 cm C18 3 μm×100 Å by Dionex/Thermo Fisher Scientific, Waltham, Mass., USA) for 5 minutes, at a flow rate of 5 μl/min. The flow was then directed in line with the Acclaim PepMap RSLC column 75 mm×50 cm C18 2 μm×100 Å (Dionex/Thermo Fisher Scientific, Waltham, Mass., USA) at a flow rate of 0.3 μl/min which is connected to the spray tip (PicoTip Emitter Silica Tip™ by New Objective, Woburn, Mass., USA). The total step gradient time was set at 104 minutes. Mobile phases A (0.1% FA, 2% ACN in water) and B (0.1% FA, 2% water in ACN) were used to create the separation gradient for eluting peptides in the column at an increasing concentration of solvent B as follows: 5% to 30% for 69 minutes; 30% to 40% for 27 minutes; 40% to 60% for 7 minutes followed by 60% to 95% for 1 minute.

For IDA (Information dependent acquisition) experiment, all data were acquired from the Triple TOF MS using Analyst TF 1.5 software by AB Sciex in the Information-dependent acquisition (IDA) mode. Peptide profiling was performed using a mass range of 350 to 1250 Da followed by a MS/MS product ion scan from 100 to 1500 Da with the abundance threshold set at more than 120 cps. The accumulation time for ions was set at 50 ms. Target ions were excluded from the scan for 12 s after being detected and former ions were excluded from the scan after one repetition. The IDA advanced ‘rolling collision energy (CE)’ option was required to automatically ramp up the CE value in the collision cell as the m/z value was increased. A maximum of 30 spectra were collected from candidate ions per cycle.

### Sample Preparation for Validation

Schirmer strips were cut into small pieces and soaked in 150 μL of 50 mM ammonium bicarbonate solution containing protease inhibitor (Halt protease inhibitor cocktail, Thermoscientific, IL, USA) for 3 hours to elute tear proteins The protein concentration was measured by colorimetric protein assay (Bio-Rad) using bovine serum album (BSA) as standard. 25 μg of tear proteins were reconstituted in 50 mM ammonium bicarbonate and reduced by Tris-(2-carboxyethyl) phosphine (TCEP). After 1.5 h of incubation at 60° C., the cysteine residue was blocked by the addition of iodoacetamide (IAA) at room temperature for 20 min. The protein sample was then digested with trypsin (trypsin:protein ratio=1:50) for 16 h at 37° C. The samples were then desalted using ultra micro spin column (The Nest Group, MA, USA).

25 μg of digested samples were reconstituted in 12 μl of loading buffer (0.1% formic acid, 2% acetonitrile in water) and 250 fmol/μl of DMBT1, LDHA, LDHB, HSPA1B and HSPB1 and 2.5 pmol/μl of ENO1 isotope-standards were spiked into the sample solution to give a final concentration of 50 fmol/μl and 500 fmol/μl respectively.

One Dimensional nanoLC-MS/MS Proteomic Analysis for Validation

RP separation was employed as described in Lei Zhou et al., Progress in Ret and Eye Res, 2012; November 31(6): 527-550, using Ultimate 3000 nanoLC system (Dionex, Thermo Fisher Scientific, MA, USA) coupled with AB Sciex 5600 triple TOF (AB Sciex, Framingham, Mass., USA) for the analysis. A 15 cm×75 μm i.d. packed with Acclaim PepMap RSLC C18 column was employed (Dionex, Thermo Fisher Scientific, MA, USA). This column was connected to a spray tip (New Objectives, Woburn, Mass.), which was directly coupled with the nano-spray interface into AB Sciex 5600 tripleTOF mass spectrometer. Samples were loaded onto a trap column (Acclaim PepMap 100 C18, 2 cm×75 μm i.d., Dionex, Thermo Fisher Scientific, MA, USA) at a flow rate of 5 μL/min. After a 5 min wash with loading buffer (2/98 v/v of ACN/water with 0.1% formic acid), the system was switched into line with the C18 analytical capillary column. A linear gradient of mobile phase B (2/98 v/v of water/ACN with 0.1% formic acid) from 7% to 30% was run for 34 minutes at flow rate of 300 nL/min was utilized for this analysis.

Third generation Nanospray Source was installed and other instrumentation settings were as follows: Ionspray Voltage Floating (ISVF)=2400 V, curtain gas (CUR)=30, Ion source gas 1 (GS1)=12, Interface Heater Temperature (IHT)=125, Declustering potential (DP)=100 V, Nebuliser current (NC)=3 for nitrogen gas. Targeted MRM approach was employed with data acquired using TOF MS+Hi Sensitivity product ion with Analyst TF 1.6 software (AB Sciex, Framingham, Mass., USA). TOF-MS scan (experiment 1) parameters were set as follows: 0.25 sec TOF MS accumulation time in the mass range of 350˜1250 Da followed by a table of respective peptide ions parameter shown in Table 3.

TABLE 3
Settings for precursor ions, collision en-
ergy (CE) and accumulation time (ms).
Heavy: isotope labeled peptide as inter-
nal standard for absolute quantitation.

| protein | m/z | CE | Acc Time ms |
|---|---|---|---|
| S100A9 | 485.913 | 32 | 100 |
| S100A11 | 530.750 | 32 | 25 |
| ENO1 | 703.860 | 40 | 150 |
| DMBT1 | 729.335 | 46 | 50 |
| LDHA | 560.324 | 34 | 100 |
| LDHB | 480.280 | 30 | 25 |
| HSPA1B | 489.276 | 32 | 50 |
| HSPB1 | 582.314 | 35 | 100 |
| LDHA Heavy | 563.833 | 34 | 100 |
| ENO1 Heavy | 706.868 | 40 | 150 |
| DMBT1 Heavy | 732.337 | 46 | 50 |
| LDHB Heavy | 483.789 | 30 | 25 |
| HSPA1B Heavy | 491.615 | 32 | 50 |
| HSPB1 Heavy | 587.318 | 35 | 100 |
| PIGR | 506.788 | 31 | 75 |
| HP | 490.750 | 31 | 20 |
| S100A8 | 636.851 | 42 | 25 |
| ORM1 | 497.764 | 31 | 200 |
| C3 | 735.893 | 46 | 30 |
| CSTB | 663.857 | 38 | 150 |
| SCGB2A1 | 690.356 | 39 | 10 |
| TIMP1 | 617.319 | 38 | 250 |
| LACRT | 593.856 | 35 | 10 |
| TGM2 | 806.410 | 45 | 150 |
| S100A4 | 445.753 | 29 | 20 |
| LCN-1 | 661.317 | 38 | 10 |
| HPX | 610.807 | 36 | 25 |
| LYZ | 700.844 | 40 | 10 |
| GSN | 915.484 | 49 | 150 |
| YWHAZ | 1021.000 | 54 | 40 |
| PIP | 642.388 | 37 | 10 |
| PROL1 | 698.901 | 40 | 20 |
| CLU | 697.352 | 40 | 20 |
| PRR4 | 801.402 | 49 | 10 |
| LTF | 1044.540 | 60 | 10 |
| MDH1 | 725.876 | 50 | 250 |
| TXN | 668.823 | 38 | 100 |
| CTSD | 521.829 | 32 | 100 |
| SOD1 | 751.384 | 42 | 150 |

The mass range was set at 100~1500 Da.

1 μl of spiked sample was injected into Ultimate 3000 nanoLC system (Dionex, Thermo Fisher Scientific, MA, USA) coupled with AB Sciex 5600 triple TOF (AB Sciex, Framingham, Mass., USA) for the analysis. 1 μl of Global Control digested tear sample (2.083 μg/μl), spiked with the same concentration as the analysed samples, was injected before every 4 sample-injection.

### Statistical Analyses

We hypothesised that tear biomarker levels would correlate with flares of SLE measured using the SLEDAI/physician global assessment. We studied this using generalised estimating equation (GEE) models with levels of each biomarker as the outcome variable. Biomarkers identified as predictors of SLE flares in the above analysis were also studied as a combined outcome variable using various combinations.

### Statistical Approach

Studied at each individual protein and combinations of proteins.

The Stats package used for data analysis was STATA v12 (Stata Corp, College Station, Tex., USA).

### Results

### Biomarker Identification

In the first stage, iTRAQ was combined with 1D nanoLC-MS/MS to screen for potential tear biomarker candidates for SLE. Eight patients with flares (each patient has two flare states, Flare 1 and Flare 2, FIG. 3) as compared with 8 SLE patients without flares and 8 age, gender and ethnicity matched controls.

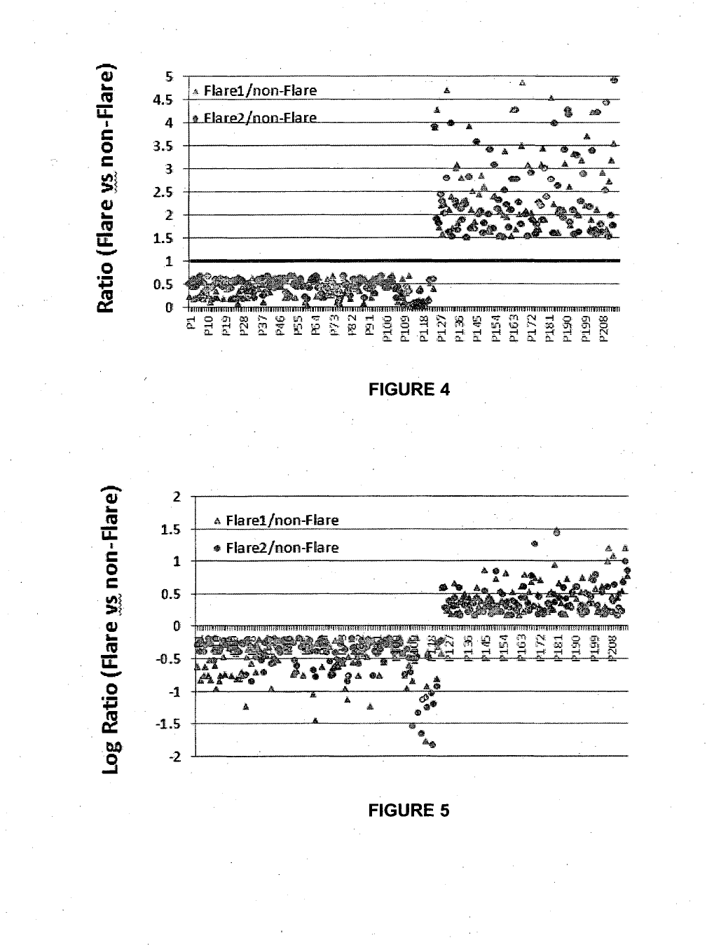

In total, 1580 tear proteins were identified [False Discovery Rate (FDR)<1%] after combing eight iTRAQ experiments. Among them, 1365 proteins were quantifiable. The expression levels of the biomarker candidates were compared as a ratio of Flare 1 and Flare 2 vs non-Flare average from 8 patients (FIG. 4), and as a log ratio (FIG. 5). Flare and non-flare states can be distinguished using a tear protein profile.

If the cutoffs for up-regulated or down-regulated were defined as >1.5 or <0.67 when Flare compared to non-Flare, 97 proteins and 123 were found to be either up-regulated or down-regulated in the Flare group as compared to the non-Flare group. Further analysis identified biomarkers alpha-Enolase (ENO1), Deleted in Brain Tumour 1 (DMBT1), Lactate dehydrogenase A (LDHA), Lactate dehydrogenase B (LDHB), Heat Shock 70 kDa Protein 1B (HSPA1B) and Heat Shock 27 kDa Protein 1 (HSPB1) as promising biomarkers.

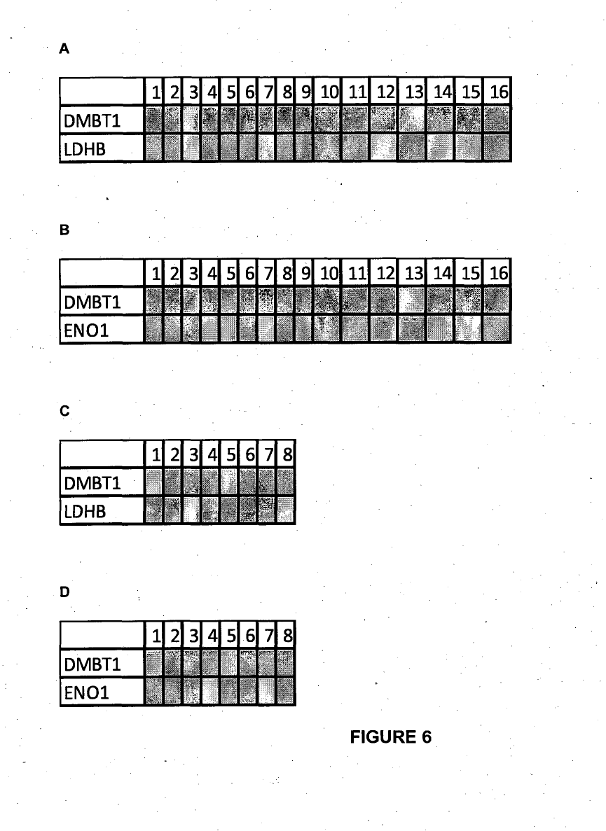

The best down-regulated protein was DMBT1. Taking a flare (8 patients with 2 samples each—at and 2 weeks after a flare) versus Non-Flare ratio <0.67, 14 out of 16 flares (87.5%) were identified correctly. The best up-regulated protein is LDHB. Using a ratio of <0.77, 15 out of 16 or 93.85% were correct (FIG. 6). With the ratio at >1.5, 11/16 (68.8%) were correctly identified. For a ratio >1.3, 12/16 (75.0%) were correct. However, the combination of 2 proteins in a panel can increase the accuracy of diagnosis (FIG. 6).

The six best tear protein biomarker candidates (DMBT1, ENO1, LDHA, LDHB, HSPA1B and HSPB1) were selected for subsequent validation in a longitudinal study.

### Validation

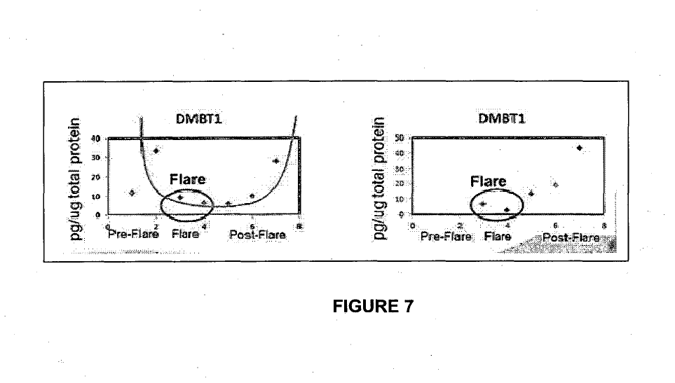

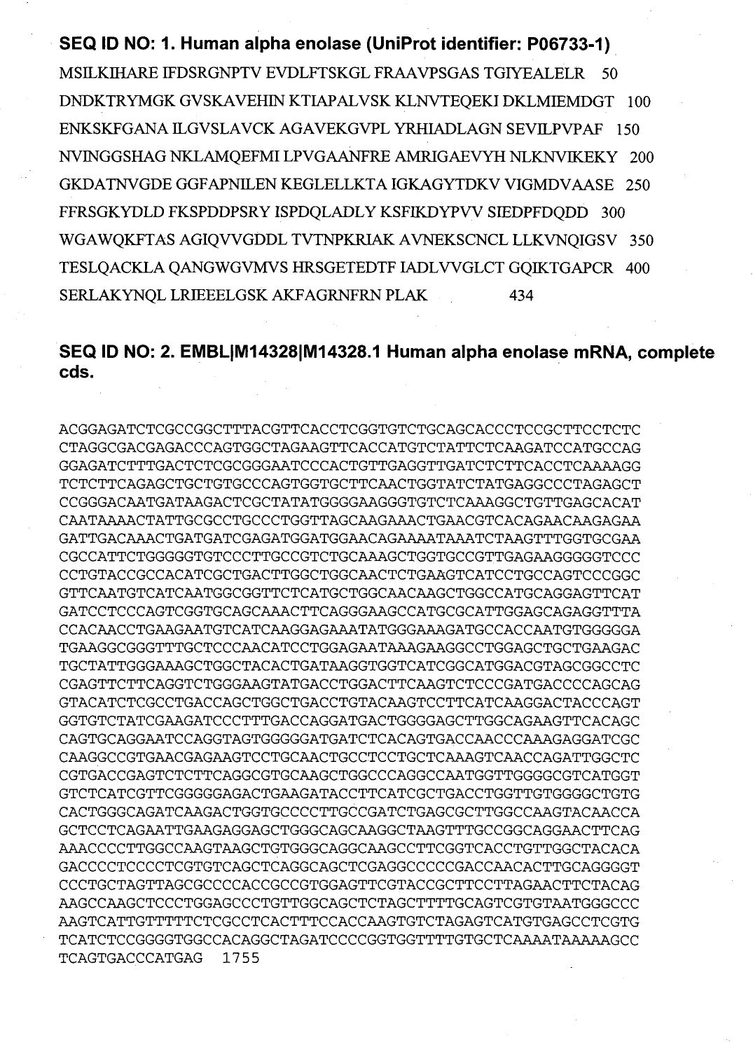

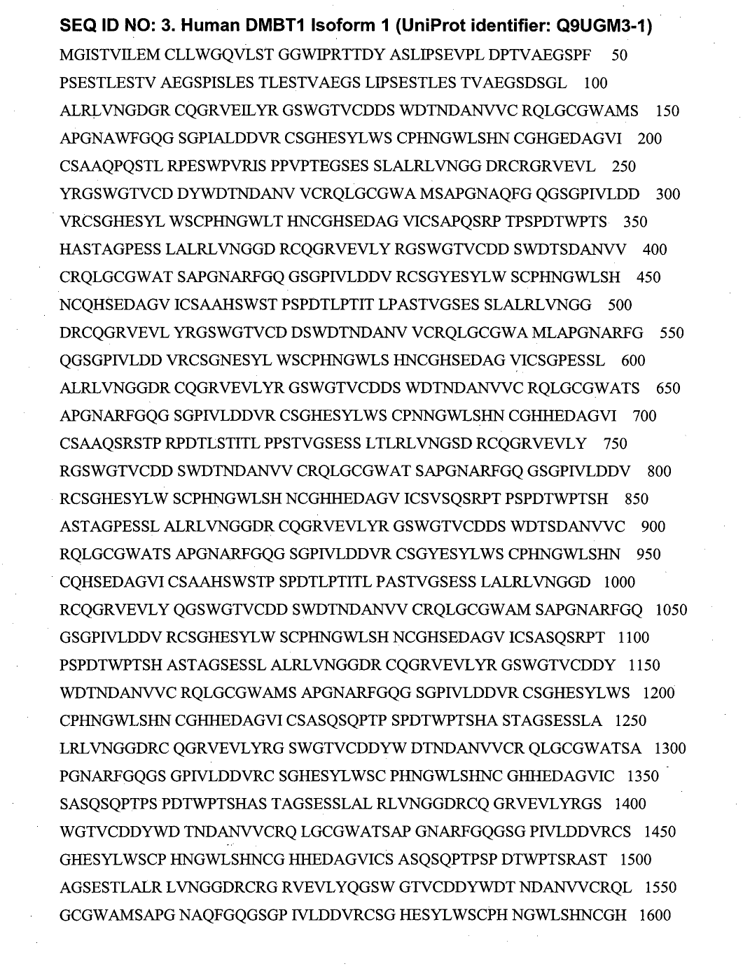

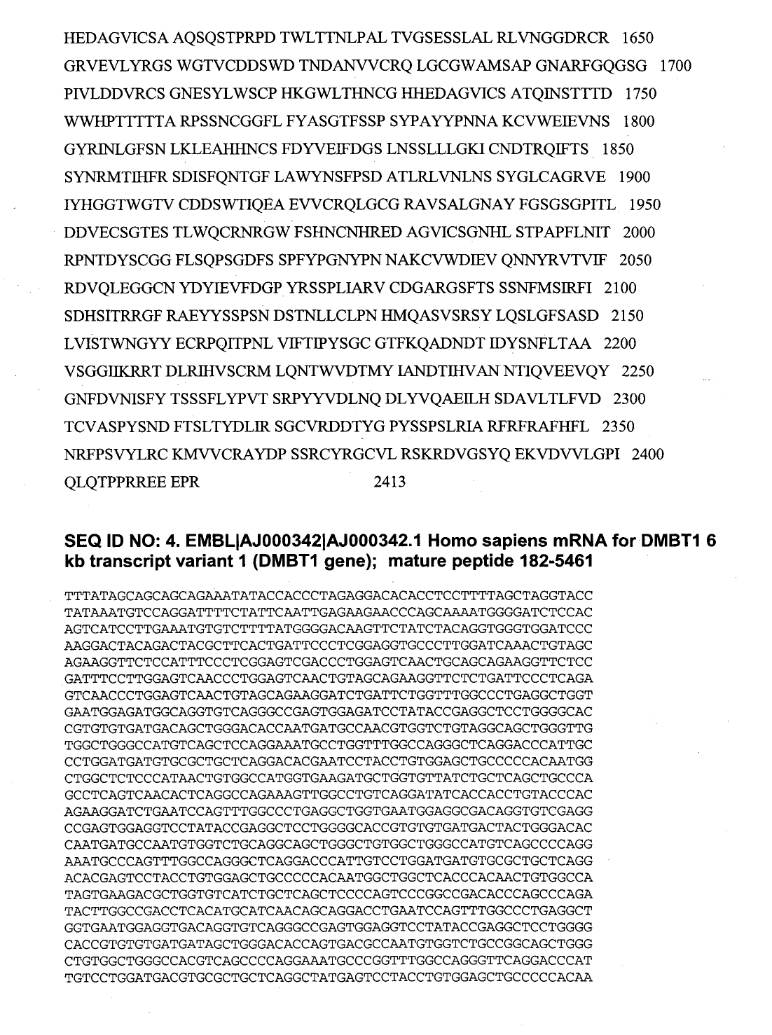

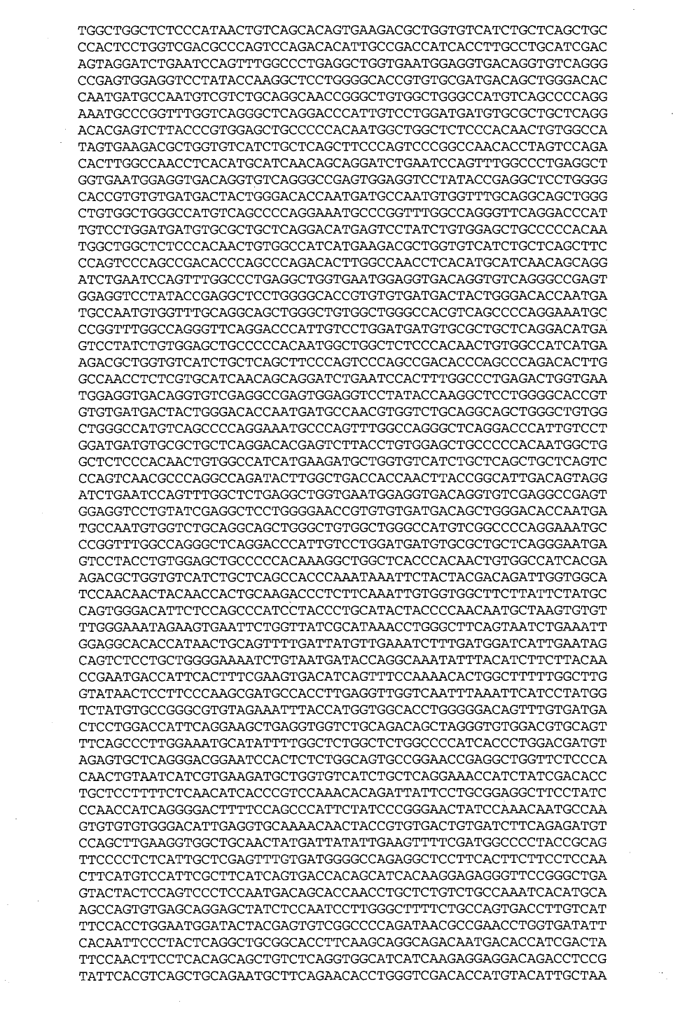

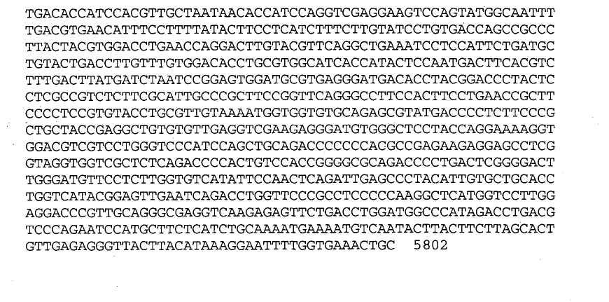

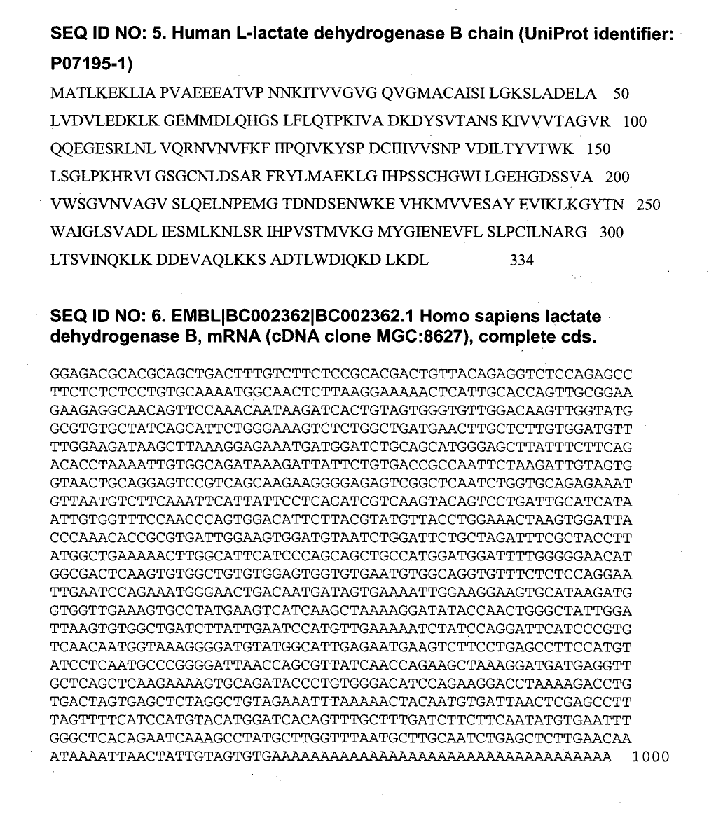

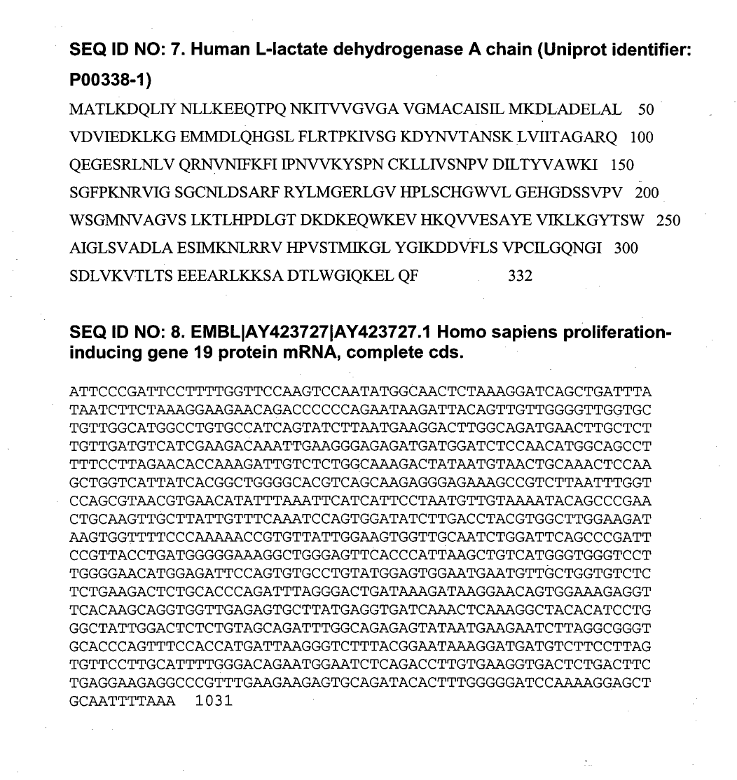

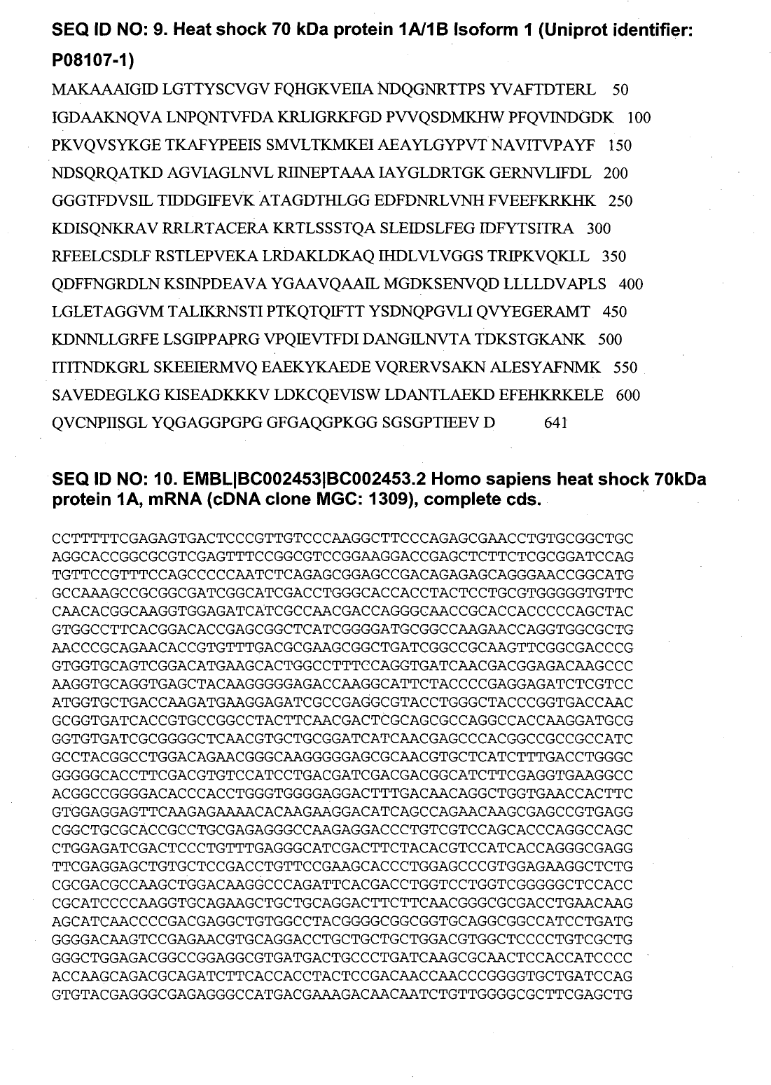

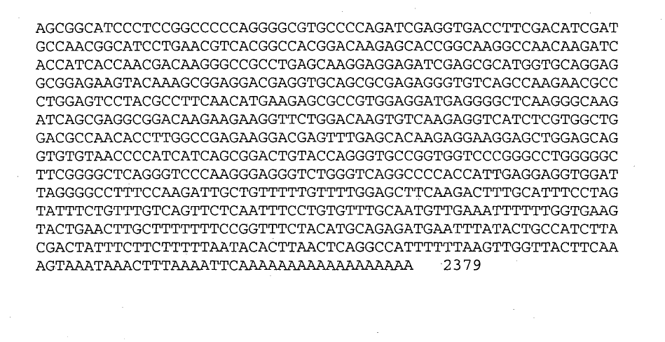

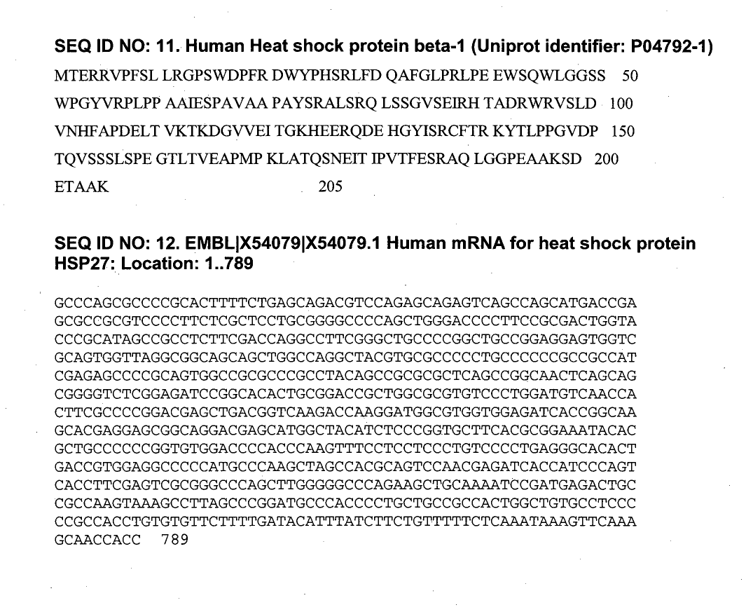

In the validation stage, High-resolution Multiple Reaction Monitoring (HR-MRM) and isotope labeled peptide standards were used to perform absolute quantitation of six tear proteins. The validation results showed that 13 out of 14 patients have at least one protein (from the 6-protein panel) matched with the expected profile which increased levels or decreased levels of proteins in flare as compared to pre-flare or post-flare. Among them, DMBT1 showed the expected profile in 12 out of 14 patients (FIG. 7).

Singly, ENO1, DMBT1 and LDHB levels are significantly differentially expressed in SLE subjects during a flare compared with after a flare (Tables 4 and 5).

TABLE 4
Description of the 6 proteins and paired comparisons

| | | | | | | | p-value | p-value | p-value |
|---|---|---|---|---|---|---|---|---|---|
| | | | | | Median value | | pre vs | post vs | Friedman |
| | Median value Pre- | | Median value at | | Post-Flare (IQR) | | Flare | Flare | test |
| Protein | Flare (IQR) (N = 7) | | Flare (IQR) (N = 14) | | (N = 10) | | (N = 7)* | (N = 10)* | (N = 3) |
| HSPB1 | 57.94 | (39.11-115.84) | 38.43 | (32.79-81.47) | 55.70 | (42.43-70.07) | 0.6121 | 0.1394 | 0.5637 |
| HSPA1B | 18.84 | (15.12-43.65) | 25.43 | (15.32-39.26) | 30.34 | (17.04-41.41) | 0.8658 | 0.0745 | 0.5637 |
| LDHA | 7.50 | (3.41-9.52) | 6.24 | (5.14-8.95) | 5.96 | (3.82-7.61) | 0.7353 | 0.9594 | 0.5637 |
| LDHB | 3.19 | (2.21-5.37) | 2.91 | (0.98-4.41) | 2.82 | (1.05-3.76) | 0.7353 | 0.0593 | 0.0833 |
| DMBT1 | 20.48 | (16.44-39.02) | 9.06 | (5.12-19.35) | 11.29 | (6.51-16.96) | 0.2367 | 0.0284 | 0.5637 |
| ENO1 | 2.59 | (2.09-4.83) | 2.60 | (1.02-4.78) | 3.11 | (1.43-4.56) | 0.8658 | 0.0218 | 0.5637 |

*Wilcoxon signed-rank test

TABLE 5
Marker of Flares identified using
Generalised Estimating Equations *

| Protein | Coef. | Std. Error | p-value |
|---|---|---|---|
| HSPB1 | −0.0013 | 0.002 | 0.424 |
| HSPA1B | −0.004 | 0.005 | 0.404 |
| LDHA | −0.023 | 0.021 | 0.265 |
| LDHB | −0.069 | 0.028 | 0.015 |
| DMBT1 | 0.006 | 0.003 | 0.058 |
| ENO1 | −0.039 | 0.030 | 0.188 |

* No adjustment for covariates because of small sample size

We have identified protein biomarkers in tear samples from SLE patients that can be used in the clinical setting to indicate the flare status of the patient.

### LIST OF REFERENCES

Abu-Sharaka M, et al: Mortality studies in systemic lupus erythematosus: results from a single centre I. Causes of death. J Rheumatol 1995; 22: 1259-64.
Ehrenstein M R, et al: The occurrence, nature and distribution of flares in a cohort of patients with systemic lupus erythematosus: a rheumatological view. Br J Rheumatol 1995; 34(3): 257-60.
Guzmán J, et al: Measurement of disease activity in systemic lupus erythematosus. Prospective validation of 3 clinical indices. J Rheumatol 1992 October; 19(10): 1551-8.
Lee L G, et al: A Low-Cost, High-Performance System for Fluorescence Lateral Flow Assays. Biosensors 2013; 3: 360-373.
Lei Zhou et al: In depth analysis of the human tear proteome. Journal of Proteomics 2012; 75: 3877-85.
Lei Zhou et al: Tear analysis in ocular surface disease. Progress in Ret and Eye Res 2012; November 31(6): 527-550.
Mirzayan M J, et al: Prognostic parameters for flare in systemic lupus erythematosus. Rheumatology (Oxford) 2000; 39(12): 1316-9.
Petri M, et al: Morbidity of systemic lupus erythematosus: role of race and socioeconomic status. Am J Med 1991; 91: 345-54.
Petri M, et al: Definition, incidence, and clinical description of flare in systemic lupus erythematosus. A prospective cohort study. Arthritis Rheum 1991; August 34(8): 937-44.
Sambrook and Russell, Molecular Cloning: A Laboratory Manual, Cold Springs Harbor Laboratory, New York (2001).
Uribe A G, et al: The Systemic Lupus Activity Measure-revised, the Mexican Systemic Lupus Erythematosus Disease Activity Index (SLEDAI), and a modified SLEDAI-2K are adequate instruments to measure disease activity in systemic lupus erythematosus. J Rheumatol 2004 October; 31(10): 1934-40.
Zhou L, et al. Identification of tear fluid biomarkers in dry eye syndrome using iTRAQ quantitative proteomics. J Proteome Res 2009; 8: 4889-905.

1. A method of analysing a fluid test sample from a subject, comprising a step of determining the level of at least one biomarker in the test sample and comparing said level to that in a reference sample, wherein the at least one biomarker is selected from the group comprising Enolase 1 (ENO1), Deleted in Brain Tumour 1 (DMBT1), Lactate dehydrogenase A (LDHA) or Lactate dehydrogenase B (LDHB) and Heat Shock 70 kDa Protein 1 B (HSPA1 B) and Heat Shock 27 kDa Protein 1 (HSPB1).

2. A method of diagnosing or monitoring the flare status of systemic lupus erythematosus (SLE) in a subject, comprising screening a fluid test sample from the subject for the presence of at least one biomarker differentially expressed in a flare state compared to a reference sample, wherein the at least one biomarker is selected from the group comprising EN01, DMBT1, LDHA, LDHB, HSPA1 B and HSPB1; and diagnosing or monitoring the status of SLE in the subject based on the expression of the at least one biomarker.

3. The method according to claim 2, wherein the reference sample represents the biomarker expression profile of a normal subject or an SLE subject in a quiescent or flare state.

4. The method according to claim 2, wherein the reference sample represents the biomarker expression profile of an SLE subject in a quiescent state.

5. The method according to claim 2, wherein the status of SLE in a subject is incipient flare or disease activity.

6. The method according to claim 2, wherein the expression of the at least one biomarker is detected using tandem Mass Spectrometry, immunoassay, or a nucleic acid technology.

7. The method according to claim 6, wherein the immunoassay is selected from the group comprising western blot, dot blot, enzyme-linked immunosorbent assay (ELISA) and lateral flow assay (LFA).

8. The method according to claim 6, wherein the nucleic acid technology further comprises hybridization or amplification in a quantitative real-time polymerase chain reaction.

9. The method according to claim 6, further comprising isolating RNA from a subject sample before the detection step.

10. The method according to claim 6, wherein detecting expression comprises using at least one primer or probe set to detect the expression of the at least one biomarker.

11. (canceled)

12. The method according to claim 2, wherein the status of SLE in a subject comprises response to a treatment administered to the subject.

13. The method according to claim 2, wherein the at least one biomarker comprises DMBT1.

14. The method according to claim 2, wherein the at least one biomarker comprises DMBT1 and EN01, or DMBT1 and LDHB.

15. The method according to claim 2, wherein the at least one biomarker is an expressed protein.

16. The method according to claim 2, wherein the test sample is a tear or saliva sample.

17. A SLE flare diagnostic or monitoring kit comprising at least one probe which specifically binds to at least one biomarker selected from the group comprising EN01, DMBT1, LDHA, LDHB, HSPAI B and HSPB1.

18. The SLE flare diagnostic or monitoring kit according to claim 17, wherein the kit comprises one or more antibodies specific to at least one of the biomarker proteins.

19. The SLE flare diagnostic or monitoring kit according to claim 18, further comprising suitable solid supports and reagents for western blot, dot blot, ELISA, lateral flow assay (LFA) or any other immunoassay platform.

20. The SLE flare diagnostic or monitoring kit according to claim 17, wherein the at least one biomarker comprises DMBT1.

21. The SLE flare diagnostic or monitoring kit according to claim 17, wherein the at least one biomarker comprises DMBT1 and EN01, or DMBT1 and LDHB.
